(19) United States
(12) Patent Application Publication
(10) Pub. No.: US 2008/0193386 A1
(43) Pub. Date: Aug. 14, 2008

(54) Compositions for Treating Alopecia or Promoting Hair Growth

(75) Inventors: Won-Min Yoo, Seoul (KR); Nae-Choon Yoo, Seoul (KR); Ki-Chang Keum, Seoul (KR); Sang-Yup Lee, Daejeon (KR); Gene Lee, Gyeonggi-do (KR)

Correspondence Address: INTELLECTUAL PROPERTY / TECHNOLOGY LAW, PO BOX 14329, RESEARCH TRIANGLE PARK, NC, 27709, US

(73) Assignee: MEDIGENES CO., LTD, Seoul (KR)

(21) Appl. No.: 11/909,461

(22) PCT Filed: Mar. 24, 2006
(86) PCT No.: PCT/KR2006/001093

§ 371 Date: Oct. 16, 2007

(30) Foreign Application Priority Data
Mar. 24, 2005 (KR) 10-2005-0024358

(51) Int. Cl. A61K 8/98; A61K 35/14; A61P 17/14; A61K 35/16

(52) U.S. Cl. 424/45; 424/529; 424/531

(57) ABSTRACT

The present invention relates to a composition for treating alopecia or promoting hair growth, which contains the blood plasma or serum as an active ingredient. The inventive composition is effective in hair growth, hair growth promotion and hair loss prevention.

### TECHNICAL FIELD

The present invention relates to a composition for inhibiting hair loss and promoting hair growth, and more particularly, to a composition for inhibiting hair loss or promoting hair growth, which contains blood plasma or serum as an active ingredient.

### BACKGROUND ART

The most common form of hair loss in men is male pattern baldness or alopecia. In the case of alopecia, hair loss occurs gradually over several years. It usually starts on the crown of the head and progresses toward the forehead area. In women suffering from alopecia, hair loss occurs in a more dispersed pattern with thinning of the hair and commonly appears following the menopause. Studies to develop a substance for alleviating or treating alopecia, particularly a substance for stimulating hair growth or reducing hair loss, have been made from long ago in the cosmetic or pharmaceutical industry field.

From the prior attempt to produce hair growth promoters, large number of compounds have been developed as candidate substances. Reported literatures, including medical, scientific and patent literatures show that there have been various efforts to treat and/or prevent alopecia and restore and/or promote hair growth, particularly with respect to human scalp hair. In fact, large number of various active compounds are suggested, and typical examples thereof may include 2,4-diamino-6-piperidinopyrimidine-3-oxide (also known as “minoxidil”) and finasteride, a specific inhibitor of type II5 Ω-reductase, which are disclosed in U.S. Pat. No. 4,139,619 and U.S. Pat. No. 4,596,812. A medicament containing minoxidil as an active ingredient is commercially available under the trademark “Rogaine” (Pharmacia & Upjohn Company). It has been suggested that Rogaine reduce hair loss up to 10% and promote hair growth in men suffering from alopecia. It is known that Rogaine is a solution for direct application to the scalp area and its therapeutic application should be continued for a long period of time. A medicament containing finasteride as an active ingredient is commercially available under the trademark “Propecia” (Merck & Co., Inc.). Propecia is a pill for oral administration. It should also be administered in a continuous and regular manner.

It has been suggested that large number of compositions based on the extracts from natural plants, including medicinal herbs, can be used for the treatment of alopecia. Various extracts of crude drugs, generally known as hair growth compositions, have been used as hair growth stimulants or promoters. However, it is common that these extracts cannot exhibit a positive effect on hair growth, because the condition of hair or alopecia is various between individuals. Even though some of these hair growth compositions show some effects, these involve defects, for example, it can be difficult to use these continuously far a long time, due to skin irritation, unpleasant odor, other side effects etc.

Another method for treatment of alopecia is hair transplantation. This method typically comprises transplanting the natural hair in the scalp area where hair grows to the bald area. However, the hair transplants often fall out after 2-4 weeks at the hair transplantation time. Although most of the hair transplants re-grow after 3-4 months, additional transplantation surgery can be required. Thus, this transplantation method requires infinitive cost and time and is painful and only limitedly successful.

Accordingly, there is an urgent need for the development of a method and a composition for hair growth, which show the excellent effects on treating alopecia or promoting hair growth over a shorter period of time, at the same time, do not involve side effects.

Accordingly, the present inventors have conducted the studies on extensive kinds of the components derived from plants and animals and, as a result, unexpectedly found that blood plasma or serum has the activity of inducing or promoting hair growth in alopecia patients, thereby completing the present invention.

### SUMMARY OF THE INVENTION

It is an object of the present invention to provide a hair growth composition and a method that show an excellent effect on treating alopecia or promoting hair growth over a shorter period of time, at the same time, do not involve side effects.

To achieve the above object, in one aspect, the present invention provides a drug or a cosmetic composition for promoting hair growth or inhibiting hair loss, which comprises the effective ingredient of blood plasma or serum as an active ingredient.

In another aspect, the present invention provides a method for preventing hair loss and promoting hair growth, the method comprises topically applying the effective ingredient of blood plasma or serum on the scalp of a subject in need thereof.

Other features and embodiments will be apparent from the following detailed description and accompanying drawings.

### BRIEF DESCRIPTION OF DRAWINGS

FIG. 1 is a photograph showing a white rat whose back has been shaved in order to test the hair growth-promoting effect of a serum-containing ointment according to the present invention. In FIG. 1, “H” represents an area to be applied with the ointment, and “C” is a control group and represents an area where hair naturally grows without application of the ointment.

FIG. 2 is a photograph showing a white rat whose back has been shaved in order to test the hair growth-promoting effect of the inventive serum-containing ointment and applied with the ointment on the half area thereof.

FIG. 3 is a photograph showing a white rat whose back has been shaved in order to test the hair growth-promoting effect of the inventive serum-containing ointment, and applied with the ointment on one side, and then covered with a transparent film so as to prevent the ointment from spreading.

FIG. 4 is a photograph showing a white rat whose back has been shaved in order to test the hair growth-promoting effect of the inventive serum-containing ointment, and applied with the ointment, and covered with a transparent film so as to prevent the ointment from spreading, and after 12 days, used to compare the relative growth of the hairs on both portions thereof.

FIG. 5 is a photograph of white rats showing hair growth-promotion results that treated the white rat with the inventive essences containing various concentrations of blood plasma.

### DETAILED DESCRIPTION OF THE INVENTION, AND PREFERRED EMBODIMENT THEREOF

The present invention relates to the use of blood plasma or serum for inhibiting hair loss or promoting hair growth. With respect to this, the present invention is characterized by topically applying a composition containing blood plasma or serum as an active ingredient to a hair loss area for about 1-8 weeks so as to promote, induce and stimulate hair growth and/or reduce hair loss. Accordingly, the composition according to the present invention is useful as an agent for treating alopecia.

As used herein, the term “inhibiting hair loss” refers to preventing, inhibiting, impeding or reducing partial or complete hair loss.

As used herein, the term “hair growth” is defined to include the maintenance, induction, stimulation, promotion and regeneration of hair growth in mammals; the growth of deficient hair; the extension of the anagen stage of the hair cycle; and the conversion of vellus hair to terminal hair.

“Alopecia” refers to deficient hair growth and partial or complete loss of hair, including, but not being limited to, androgenic alopecia (male pattern baldness), toxic alopecia, alopecia areata, telogen effluvium, alopecia caused by endocrine abnormality, metabolic disorder and nutritional disorder, drug-induced alopecia, mechanical alopecia, alopecia induced by skin disease, alopecia cicatrisata, congenital alopecia, and trichotillomania. Alopecia occurs when the pilar cycle is disturbed. The most frequent phenomenon is a shortening of the hair's growth cycle or anagen phase due to cessation of cell proliferation. This results in early onset of the catagen phase, and consequently a large number of hairs in the telogen phase during which the hair follicles are detached from the dermal papillae, and the hairs fall out. Alopecia has large number of etiologies, including genetic factors, aging, local and systemic diseases, febrile conditions, mental stresses, hormonal problems, and secondary effects of drugs.

As used herein, the term “treating alopecia” refers to: preventing alopecia in an animal which may be predisposed to alopecia; and/or inhibiting, retarding or reducing alopecia; and/or promoting hair growth, and/or prolonging the anagen phase of the hair cycle, and/or converting vellus hair into terminal hair. Terminal hair is coarse, pigmented, long hair in which the bulb of the hair follicle is seated deeply in the dermis. Vellus hair, on the other hand, is fine, thin, non-pigmented short hair in which the hair bulb is located superficially in the dermis. As alopecia progresses, the hairs change from the terminal to the vellus type.

Blood plasma which is used as an active ingredient in the present invention typically refers to a light yellow-colored liquid component from which solid components (i.e., cells and cell fragments) in mammalian blood have been separated, and the components and composition thereof is well known, for example, in the following literature: Philip Westerman, Plasma Proteins, VII-1 to VII-13, Sep. 17, 2002; and Wendy Y. Craig, et al., Plasma Proteins Pocket Guide, Foundation for Blood Research, the entire disclosure thereof is incorporated herein by reference. Serum is also well defined and generally referred to one obtained by removing fibrinogen and other coagulation factors from blood plasma.

In the present invention, a source for supplying blood plasma or serum encompasses all species of mammals, including human and non-human primates, for example, domestic animals, such as sheep, goats, pigs, horses, dogs and cattle, and other primates and rodents.

In the present invention, blood plasma or serum can be easily separated from blood according to any conventional method in the art, for example, centrifugation, settlement or filtration processes. The centrifugation process may be carried out under conditions suitable for precipitating blood cells from plasma. For example, the centrifugation process is carried out at 3,000 rpm for 10 minutes, and these conditions are sufficient to precipitate not only red blood cells and white blood cells, but also substantially all cell fragments (platelets).

The supernatant containing blood plasma can be easily separated from the precipitated cells according to the standard technology. The filtration process can be performed by passing blood through a filter suitable for separating blood cells from blood plasma. The filter may be a microporous membrane allowing good permeation of proteins.

It is known that blood plasma or serum is stored before use as fresh liquid plasma or liquid preparations obtained by centrifugation or sedimentation after blood collection, and is also stored as various forms, such as fresh frozen preparations, cryoprecipitates, freeze-dried preparations or concentrated preparations. Plasma or serum in these conditions can all be used in the present invention. The fresh frozen plasma is prepared by centrifuging blood at about 2800 rpm for about 15 minutes within 6 hours after blood collection to separate blood cells and a plasma compounds from the blood and freezing the plasma component at the temperature range from −18° C. to −40° C. It is used after thawing in hot water the temperature range of 30-37° C.

The cryoprecipitated plasma is prepared by dissolving one unit of fresh frozen plasma at about 4° C., separating the resulting cold precipitated proteins (including a large amount of factors, such as VIII:C, fibrinogen, XIII, fibronectin, etc.), and refreezing the separated proteins at a temperature ranging from −18° C. to −40° C. The cryoprecipitates can be used after thawing them in a refrigerator at 1-6° C. overnight or more rapidly thawing them in water bath at about 4° C. The concentrated plasma is obtained by separating plasma from blood, mixing the separated plasma with a concentrating agent, such as dextranomer, Sephadex, destramine, polyacrylamide, bio-gel P, silica gel, zeolite, Debrisan, crosslinked agarose, starch or alginate gel, so as to concentrate the plasma, and separating the concentrating agent from the concentrate.

In one embodiment of the present invention, commercially available blood plasma or serum may be used. For example, the following plasma or serum may be used: a powdery preparation commercially available from the Blood Bank, liquid preparations commercially available from Invitrogen Corporation (e.g., Gibco™ Chichen Serum, Gibco™ Goat Serum, Gibco™ Lamb Serum, Gibco™ Porcine Serum, and Gibco™ Rabbit Serum), and serum preparations commercially available from Gemini Bio-Products (CA 95776, USA) (e.g., Chicken Serum (Cat.#100-161), Dog Serum (Cat.#100-160), Donor Donkey Serum (Cat.#100-151), Donor Goat Serum(Cat.#100-109), Donor Rat Serum(Cat.#100-155), Feline Serum(Cat.100-153), Guinea Pig Serum(Cat.#100-130), Monkey Serum(Cat.#100-154), Mouse Serum(Cat#100-113), Porcine Serum(Cat.#100-115), Rabbit Serum(Cat.#100-116), Rat Serum(Cat.#100-150), Sheep Serum(Cat.#100-117)). These products are derived from serum units of human and other animal origins, and test results showed that these products are non-reactive to various antigens and antibodies, for example, hepatitis B surface antigen (HBsAg) and hepatitis C virus (HCV) antibody and are negative for antibodies to HIV-1 and HIV-2. All plasma units which are used for the production of these preparations have previously been proven to be nonpathogenic. To reduce the latent risk of propagation of pathogenic bacteria, the preparations can be treated with an organic solvent/cleaner mixture, such as tri(n-butyl)/phosphate/polysorbate 80 designed to inactivate envelop viruses, such as HIV, hepatitis B virus and HCV. Also, the removal of virus can be enhanced by additionally performing a nanofiltration step.

In another embodiment, the preparations can be prepared using an independent purification technique (i.e., solvent detergent and nanofiltraton) versus pasteurization. The purification may be carried out in the state of blood or plasma

The produced blood plasma or serum fraction can be powdered through heating, freeze-drying or other suitable drying techniques. For example, blood plasma or serum can be freeze-dried at a temperature of less than −40° C. for several days (e.g., about 7 days). Conventional techniques and parameters known to those skilled in the art can be used.

To effectively treat alopecia or effectively promote hair growth, blood plasma or serum used in the inventive method and drug composition should easily act at a target site. For this purpose, a composition suitable for use according to the present invention includes a preparation containing blood plasma or serum as an active ingredient in combination with a pharmaceutically acceptable carrier.

Blood plasma or serum is present in an amount of 0.1-99.9 wt % based on the weight of the composition. Alternatively, blood plasma or serum can also be used alone without the carrier. As used herein, the term “pharmaceutically acceptable carrier” means a carrier that is miscible with other components of the preparation and does not adversely affect a person receiving it.

Typically, blood plasma or serum is topically administered. Formulations suitable for topical administration include liquid or semi-liquid formulations, such as lotion, emulsion, cream, ointment, liniment, spray, aerosol, oil, paste, gel, tonic, solution or suspension. These hair growth formulations can be prepared by mixing and dissolving various components or kneading the mixture using any apparatus or method which is conventionally used or well known in the pharmaceutical and/or cosmetic technology field [Remington's Pharmaceutical Science, 15th Edition, 1975, Mack Publishing Company, Easton, Pa. 18042 (Chapter 87: Blaug, Seymour)]. Preferred formulations are ointment, lotion and cream.

For ointments, blood plasma or serum is suspended or dissolved in one or a mixture of two or more selected from the following substances: mineral oil, paraffin, inorganic oil, white Vaseline, propylene glycol, polyoxyethylene, polyoxypropylene, glycerin, stearyl alcohol, emulsifying wax, cetanol, sodium lauryl sulfate, ethyl or butyl paraoxybenzoate, saline and water. For lotions or creams, blood plasma or serum is suspended or dissolved in one or a mixture of two or more selected from among the following substances: mineral oil, sorbitan monostearate, polysorbate 60, Vaseline, lanolin, saline, cetyl ester wax, cetearyl alcohol, 2-octyl dodecanol, benzyl alcohol and water.

The formulation according to the present invention may comprise, in addition to the above-described components, at least one additional component selected among diluents, buffers, flavoring agents, binders, surfactants, thickeners, lubricants, preservatives, pH adjusting agents, sterilizing agents, antioxidants, emulsifiers, stabilizers, fragrances and colorants.

The dosage of blood plasma or serum according to the present invention should be suitably determined considering sex, age, hair loss conditions, hair conditions, etc. For normal adults, blood plasma or serum is applied to the scalp at a dosage of about 0.1-5 mg/cm²/day.

### EXAMPLES

Hereinafter, the present invention will be described in further detail with reference to examples. However, it will be obvious to those skilled in the art that these examples are presented to more fully describe the present invention and are not to be construed to limit the scope of the present invention.

### Example 1

### Pre-Paration of Ointment Containing Human Blood Plasma

A blood preparation (fresh frozen plasma, Central Blood Center, Korea) derived from persons who have been identified to be negative for potential pathogenic viruses, including HIV, HCV and HBV, was thawed at 30° C., and 500 ml of the blood sample was placed in a freeze-drying bottle and then frozen in a deep freezer (Forma Science, Inc. Ohio, USA) at −80° C. for 8 hours. The frozen bottle was placed in a freeze dry/shell freeze system (Labconco, co. Kansas City, Mo., USA) and freeze-dried at −48° C. for 7 days while operating the system. All procedures were performed in sterile condition 500 ml of blood plasma provided about 30 g of plasma powder.

5 g of the above-prepared powder was mixed with 95 g of semibase cream (SAM-A Pharmaceutical Industrial Co., Ltd, Korea), an water soluble ointment base, to which a suitable amount of physiological saline was then added. To the mixture, 1N HCl or 1N NaOH was added and stirred, at the same time, measured for pH with a pH meter (Orion), thus preparing an ointment preparation having an adjusted pH of 5.5. The ointment base consisted of 38 mg of hard lead, 116 mg of stearyl alcohol, 38 mg of polyethylene glycol 4000, 192 mg of concentrated glycerin, 23 mg of cetanol, a suitable amount of purified water, 9 mg of sodium lauryl sulfate, 0.87 mg of ethyl paraoxybenzoate and 0.12 g of butyl paraoxybenzoate based on 1 g of the ointment base.

### Example 2

### Preparation of Ointment Containing Bovine Serum

500 ml of fetal bovine serum (FBS; Biofluids. Inc, Rockville, Md.) was placed in a freeze-drying bottle and frozen in a deep freezer (Forma Scientific, Inc. Ohio, USA) at −80° C. for 6 hours. The above used FBS had endotoxin level of less than 0.1 ng/ml and a hemoglobin level of less than 30 ng/100 ml. The bottle containing the frozen FBS was placed in a freeze dry/shell freeze system (Labconco, Co. Kansas City, Mo. USA) and freeze-dried at −48° C. for 7 days while operating the system, thus preparing a powder preparation. All the above procedures were carried out under sterile conditions.

5 g of the above-prepared powder was mixed with 95 g of semibase cream (SAM-A Pharmaceutical Industrial Co., Ltd, Korea), an water soluble ointment base, to which a suitable amount of physiological saline was then added. To the mixture, 1N HCl or 1N NaOH was added and stirred, at the same time, measured for pH with a pH meter (Orion), thus preparing an ointment preparation having an adjusted pH of 5.5.

### Example 3

### Preparation of Gel Containing Human Blood Plasma

5 parts by weight of the powder prepared in Example 1 was mixed with 95 parts by weight of components (38 mg of Carbopol ETD 2020, 116 mg of glycerin, 38 mg of propylene glycol, 192 mg of triethanolamine and a suitable amount of purified water), thus obtaining a gel preparation having a pH of 5.8-6.0. Carbopol ETD 2020 is an acrylate having a C₁₀₋₃₀ alkyl acrylate crosspolymer.

### Example 4

### Preparation of Essence Containing Porcine Blood Plasma

The composition of an essence prepared in this Example is shown in Table 1 below.

TABLE 1

| No. | Materials | Amount (wt. %) |
|---|---|---|
| 1 | distilled water | 15.00 |
| 2 | porcine blood plasma | 0.5, 1.0, 1.5, 2.0 or 2.5 |
| 3 | Methylchloroisothiazolin | 0.05 |
| 4 | Buthyleneglycol | 9.00 |
| 5 | Menthol | 0.22 |
| 6 | tween 80 | 0.35 |
| 7 | flavor (H-312221) | 0.07 |
| 8 | Phnoxyethanol | 0.25 |
| 9 | distilled water | 1.0 |
| 10 | ketrol-F (2%) | 0.2 |
| 11 | distilled water | 8 |
| 12 | hydroxypropylethyl cellulose | 0.08 |
| 13 | distilled water | 8 |
| 14 | sepigel-305 | 0.85 |
| 15 | distilled water | 100% residue |

Material Nos. 2 and 3 were completely dissolved in material No. 1 and fed into a main tank. In a separate process, material Nos. 4-9 were weighed, completely dissolved, introduced into the tank and sufficiently stirred. Material No. 11 was dissolved in material No. 10, and the solution was introduced into the tank and stirred. Material No. 13 was dissolved in material No. 12, and the solution was introduced into the main tank and stirred. The solution in the tank was heated to 50° C. Material No. 14 was introduced into the tank with homomixing, and the solution in the tank was emulsified. The emulsification process was carried out for 5 minutes at 3600 rpm and a pad speed of 200 rpm, and then material No. 15 was added in an amount up to 100 wt %. Then, the solution in the tank was cooled to 30° C., thus obtaining essences containing various concentrations of blood plasma.

### Example 5

### Hair Growth-Promoting Effect of Bovine Serum

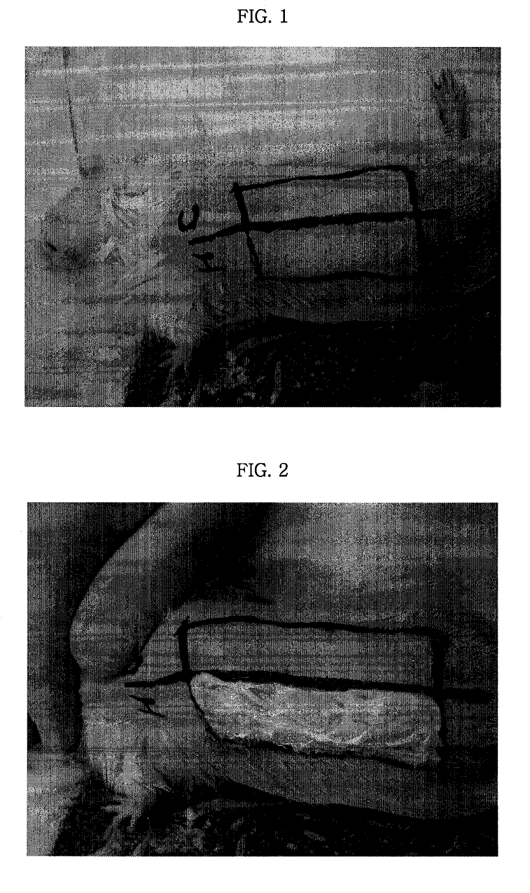

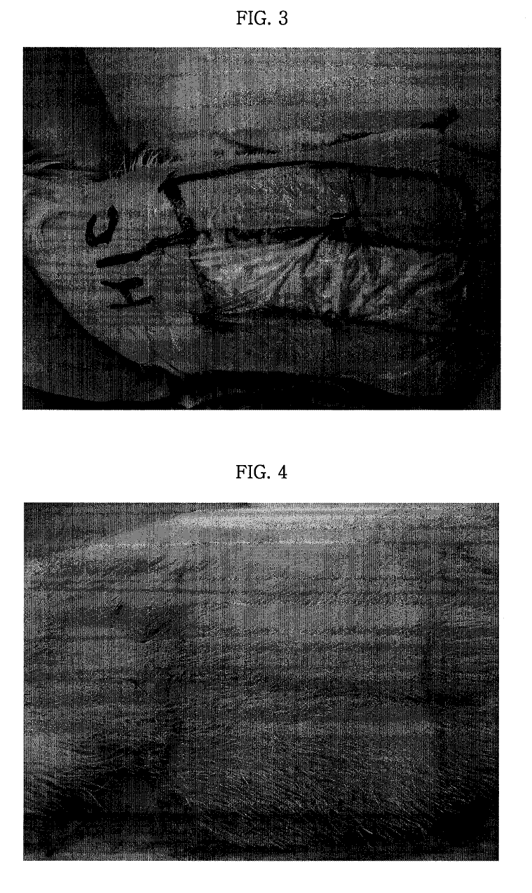

The back of each of ten mature white rats (300-350 mg, Sprague-Dawley) was shaved and bisected with a black pen. One-half the area of the back was applied with the ointment prepared in Example 2, and the other half area remained untouched. In this state, the back applied with the ointment was covered with a transparent film having a size slightly larger than the shaved area so as to prevent the ointment from spreading laterally. The transparent film was fixed by sewing with sutures (see FIGS. 1˜3). After 12 days, the film was removed, and the back was observed for hair growth. It was observed that, in all the tested white rats, the hairs grown on the back area applied with the ointment were remarkably thicker and larger in number than the hairs naturally grown on the back area not applied with the ointment (FIG. 4).

### Example 6

### Hair Growth-Promoting Effect of Porcine Blood Plasma

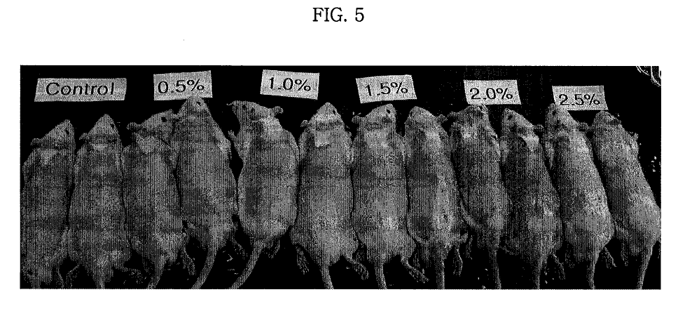

(1) Twenty-five 4-week-old S/D (Spraue-Dawely) white rats were selected and the back of each animal was shaved, and then uniformly applied with a hair remover (commercially available under the trademark “Niclean Cream” from I1-Dong Pharmaceutical Co., Ltd.) in an amount of 20-30 g/animal. (2) After 20 minutes, the cream was removed with flowing water while removing the hair. (3) On the area from which the hair has been removed, testosterone (commercially available under the trademark “Testo” from Sam-Il Pharmaceutical Co., Ltd., Korea; ethanol-Merk) in ethanol was applied in an amount of 50 mg/2 ml/animal daily for 3 weeks. (4) During the step (3), 5 mg/animal of testosterone was subcutaneously injected at threeday intervals. (5) After 3 weeks of treatment with testosterone, 12 white rats whose back had no hair grown thereon were selected and subjected to the steps (1) and (2). (6) The test animals were divided into the groups consisting of 2 animals respectively, and the back of each of the test animals was treated with varying plasma concentrations (0% (control group, physiological saline), 0.5%, 1.0%, 1.5%, 2.0% and 2.5%) of the essence prepared in Example 4, in an amount of 2 m/animal daily for 3 weeks. (7) After 3 weeks, the test animal of each of the test group was taken and photographed (see FIG. 5), 20-30 hairs grown on the back of each test animal were randomly taken and placed on a slide glass, onto which one drop of distilled water was dropped. Then, the hairs on the slide glass was covered with a cover glass and observed at 100× magnification, and their thickness was measured with a scale on the microscope. The measurement results are shown in Table 2 below.

TABLE 2

| | | | | | | | | | | | | Standard |
|---|---|---|---|---|---|---|---|---|---|---|---|---|
| Conc. | S1 | S2 | S3 | S4 | S5 | S6 | S7 | S8 | S9 | S10 | Average | deviation |
| Control | 45 | 44 | 57 | 42 | 48 | 48 | 53 | 48 | 59 | 54 | 49.8 | 5.6920998 |
| 0.5 | 44 | 42 | 43 | 42 | 48 | 59 | 60 | 48 | 47 | 48 | 48.1 | 6.4884513 |
| 1 | 70 | 68 | 69 | 77 | 58 | 54 | 99 | 78 | 77 | 73 | 72.3 | 12.311241 |
| 1.5 | 88 | 99 | 90 | 66 | 87 | 103 | 79 | 153 | 105 | 103 | 97.3 | 23.070183 |
| 2 | 155 | 156 | 122 | 135 | 157 | 158 | 188 | 175 | 153 | 165 | 156.4 | 18.488435 |
| 2.5 | 157 | 185 | 132 | 157 | 158 | 188 | 122 | 195 | 177 | 163 | 163.4 | 23.726216 |

### INDUSTRIAL APPLICABILITY

As described above in detail, the inventive composition containing blood plasma or serum is effective in hair growth, hair growth promotion and hair loss prevention. Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A pharmaceutical composition for promoting hair growth or inhibiting hair loss comprising an effective amount of blood plasma or serum as an active ingredient.

2. The pharmaceutical composition for promoting hair growth or inhibiting hair loss according to claim 1, wherein said blood plasma or serum is freeze-dried form or powder.

3. The pharmaceutical composition for promoting hair growth or inhibiting hair loss according to claim 1, wherein said blood plasma or serum is originated from human, non-human primates or mammals.

4. The pharmaceutical composition for promoting hair growth or inhibiting hair loss according to claim 1, wherein the blood plasma or serum is present alone or in combination with a pharmaceutically acceptable carrier.

5. The pharmaceutical composition for promoting hair growth or inhibiting hair loss according to claim 4, wherein the pharmaceutically acceptable carrier includes a topical carrier.

6. The pharmaceutical composition for promoting hair growth or inhibiting hair loss according to claim 5, wherein the formulation is for topical administration, selected from the group consisting of cream, lotion, tonic, spray, aerosol, oil, solution, emulsion, gel, and ointment.

7. The pharmaceutical composition for promoting hair growth or inhibiting hair loss according to claim 5, wherein the content of said blood plasma or serum is 0.1-99.9 wt % based on the weight of the composition.

8. The pharmaceutical composition for promoting hair growth or inhibiting hair loss according to claim 5, which additionally comprises one or more adjuvant selected from the group consisting of a diluent, a buffer, a flavoring agent, a binder, a surfactant, a thickener, a lubricant, a preservative, an antimicrobial preserving agent, an antioxidant, and emulsifiers.

9. A cosmetic composition for promoting hair growth or inhibiting hair loss comprising an effective amount of blood plasma or serum as an active ingredient.

10. The cosmetic composition for promoting hair growth or inhibiting hair loss according to claim 9, wherein said blood plasma or serum is freeze-dried form or powder.

11. The cosmetic composition for promoting hair growth or inhibiting hair loss according to claim 9, wherein said blood plasma or serum is originated from human, non-human primates or mammals.

12. The cosmetic composition for promoting hair growth or inhibiting hair loss according to claim 9, wherein the blood plasma or serum is present alone or in combination with a pharmaceutically acceptable carrier.

13. The cosmetic composition for promoting hair growth or inhibiting hair loss according to claim 12, wherein the pharmaceutically acceptable carrier includes a topical carrier.

14. The cosmetic composition for promoting hair growth or inhibiting hair loss according to claim 13, wherein the formulation is for topical administration, selected from the group consisting of cream, lotion, tonic, spray, aerosol, oil, solution, emulsion, gel, and ointment.

15. The cosmetic composition for promoting hair growth or inhibiting hair loss according to claim 13, wherein the content of said blood plasma or serum is 0.1-99.9 wt % based on the weight of the composition.

16. The cosmetic composition for promoting hair growth or inhibiting hair loss according to claim 13, which additionally comprises one or more adjuvant selected from the group consisting of a diluent, a buffer, a flavoring agent, a binder, a surfactant, a thickener, a lubricant, a preservative, an antimicrobial preserving agent, an antioxidant, and emulsifiers.

17. A method for promoting hair growth, the method comprising topically applying the composition for promoting hair growth of claim 1 to the skin of a mammal.

18. A method for preventing hair loss, the method comprising topically applying the composition for promoting hair growth of claim 1 to the skin of a mammal.
